(19) United States
(12) Patent Application Publication
(10) Pub. No.: US 2008/0261975 A1
(43) Pub. Date: Oct. 23, 2008

(54) Tec Kinase Inhibitors

(76) Inventors: Joerg Martin Bentzien, Tarrytown, NY (US); Hidenori Takahashi, Lagrangeville, NY (US)

Correspondence Address: MICHAEL P. MORRIS;BOEHRINGER INGELHEIM USA CORPORATION, 900 RIDGEBURY ROAD, P O BOX 368, RIDGEFIELD, CT, 06877-0368, US

(21) Appl. No.: 12/092,751

(22) PCT Filed: Nov. 7, 2006
(86) PCT No.: PCT/US06/43386

§ 371 Date: May 6, 2008

Related U.S. Application Data
(60) Provisional application No. 60/735,471, filed on Nov. 12, 2005.

(51) Int. Cl. A61K 31/5377; C07D 471/04; A61P 35/00; A61K 31/437

(52) U.S. Cl. 514/234.5; 546/113; 514/300; 544/127

(57) ABSTRACT

Disclosed are compounds of formula (I):

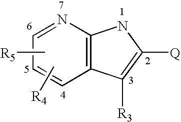

wherein Q, R₁, R₂, R₃, R₄ and R₅ are defined herein. The compounds of the invention inhibit Itk kinase and are therefore useful for treating diseases and pathological conditions involving inflammation, immunological disorders and allergic disorders. Also disclosed are processes for preparing these compounds and to pharmaceutical compositions comprising these compounds.

### APPLICATION DATA

This application is a 371 National Stage of PCT application PCT/US2006/043386, filed Nov. 7, 2006, which claims benefit to U.S. provisional application Ser. No. 60/735,471 filed Nov. 12, 2005.

### TECHNICAL FIELD OF THE INVENTION

This invention relates to substituted pyrrolo[2,3-b]pyridine compounds of formula (I):

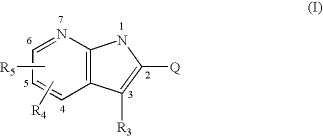

wherein Q, R₃, R₄, R₅ are defined herein below. The compounds of the invention inhibit Itk kinase and are therefore useful for treating diseases and pathological conditions involving inflammation, immunological disorders and allergic disorders. This invention also relates to processes for preparing these compounds and to pharmaceutical compositions comprising these compounds.

### BACKGROUND OF THE INVENTION

Protein kinases play a critical role in mediating signaling events leading to cellular responses such as activation, growth and differentiation, in response to extracellular signals. Protein kinases transmit their signal by phosphorylating specific residues in a target protein. Protein kinases that specifically phosphorylate tyrosine residues are referred to as protein tyrosine kinases. Protein tyrosine kinases can be divided into two general groups: receptor such as epidermal growth factor (EGF) receptor (S. Iwashita and M. Kobayashi, 1992, Cellular Signalling, 4, 123-132) and cytosolic non-receptor (C. Chan et al., 1994, Ann. Rev. Immunol., 12, 555-592).

Interleukin-2-inducible T cell kinase (Itk), also referred to as T cell-specific kinase (Tsk) and expressed mainly in T-lymphocytes (EMT), is a member of the Tec family of protein tyrosine kinases that also includes Txk, Tec, Btk, and Bmx. Tec family members are characterized by the presence of a pleckstrin-homology domain (PH), a proline rich Tec homology domain (TH) and Src homology SH3, SH2 and SH1 kinase domains positioned from the N-terminus to the C-terminus respectively (S. Gibson et al., 1993, Blood, 82, 1561-1572; J. D. Siliciano et al., 1992, Proc. Nat. Acad. Sci., 89, 11194-11198; N. Yamada et al., 1993 Biochem. and Biophys Res. Comm., 192, 231-240).

Itk is expressed in T cells, mast cells and natural killer cells. It is activated in T cells upon stimulation of the T cell receptor (TCR), and in mast cells upon activation of the high affinity IgE receptor. Following receptor stimulation in T cells, Lck, a src tyrosine kinase family member, phosphorylates Y511 in the kinase domain activation loop of Itk (S. D. Heyeck et al., 1997, J. Biol. Chem., 272, 25401-25408). Activated Itk, together with Zap-70 is required for phosphorylation and activation of PLC-γ (S. C. Bunnell et al., 2000, J. Biol. Chem., 275, 2219-2230). PLC-γ catalyzes the formation of inositol 1,4,5-triphosphate and diacylglycerol, leading to calcium mobilization and PKC activation, respectively. These events activate numerous downstream pathways and lead ultimately to degranulation (mast cells) and cytokine gene expression (T cells) (Y. Kawakami et al., 1999, J. Leukocyte Biol., 65, 286-290).

The role of Itk in T cell activation has been confirmed in Itk knockout mice. CD4⁺ T cells from Itk knockout mice have a diminished proliferative response in a mixed lymphocyte reaction or upon Con A or anti-CD3 stimulation. (X. C. Liao and D. R. Littman, 1995, Immunity, 3, 757-769). Also, T cells from Itk knockout mice produced little IL-2 upon TCR stimulation resulting in reduced proliferation of these cells. In another study, Itk deficient CD4⁺ T cells produced reduced levels of cytokines including IL-4, IL-5 and IL-13 upon stimulation of the TCR, even after priming with inducing conditions. (D. J. Fowell, 1999, Immunity, 11, 399-409).

The role of Itk in PLC-γ activation and in calcium mobilization was also confirmed in the T cells of these knockout mice, which had severely impaired IP₃ generation and no extracellular calcium influx upon TCR stimulation (K. Liu et al., 1998, J. Exp. Med. 187, 1721-1727). The studies described above support a key role for Itk in activation of T cells and mast cells. Thus an inhibitor of Itk would be of therapeutic benefit in diseases mediated by inappropriate activation of these cells.

It has been well established that T cells play an important role in regulating the immune response (Powrie and Coffman, 1993, Immunology Today, 14, 270-274). Indeed, activation of T cells is often the initiating event in immunological disorders. Following activation of the TCR, there is an influx of calcium that is required for T cell activation. Upon activation, T cells produce cytokines, including IL-2,4, 5, 9, 10, and 13 leading to T cell proliferation, differentiation, and effector function. Clinical studies with inhibitors of IL-2 have shown that interference with T cell activation and proliferation effectively suppresses immune response in vivo (Waldmann, 1993, Immunology Today, 14, 264-270). Accordingly, agents that inhibit T lymphocyte activation and subsequent cytokine production, are therapeutically useful for selectively suppressing the immune response in a patient in need of such immunosuppression.

Mast cells play a critical roll in asthma and allergic disorders by releasing pro-inflammatory mediators and cytokines. Antigen-mediated aggregation of FcεRI, the high-affinity receptor for IgE results in activation of mast cells (D. B. Corry et al., 1999, Nature, 402, B18-23). This triggers a series of signaling events resulting in the release of mediators, including histamine, proteases, leukotrienes and cytokines (J. R. Gordon et al., 1990, Immunology Today, 11, 458-464.) These mediators cause increased vascular permeability, mucus production, bronchoconstriction, tissue degradation and inflammation thus playing key roles in the etiology and symptoms of asthma and allergic disorders.

Recent published data using Itk knockout mice suggests that in the absence of Itk function, increased numbers of memory T cells are generated (A. T. Miller et al., 2002 The Journal of Immunology, 168, 2163-2172). One strategy to improve vaccination methods is to increase the number of memory T cells generated (S. M. Kaech et al., Nature Reviews Immunology, 2, 251-262).

All patent and literature documents cited in this application are incorporated by reference in their entirety.

### SUMMARY OF THE INVENTION

It is therefore an object of the invention to provide a compound of the formula (I):

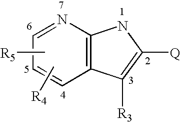

wherein Q, R₃, R₄, R₅ are defined herein below.

It is another object of the invention to provide a method of inhibiting the Tec kinase family, including Itk kinase, and methods of treating diseases or conditions related to such kinase activity activity, by administering to a patient in need thereof a therapeutically effective amount of a compound of the formula (I). It is yet another object of the invention to provide pharmaceutical compositions and processes of making compounds of the formula (I) as described herein below.

### DETAILED DESCRIPTION OF THE PREFERRED EMBODIMENTS

In it's broadest generic embodiment, the invention provides for a compound of the formula (I):

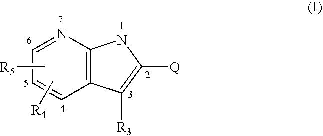

wherein:

Q is

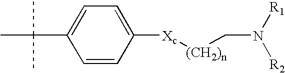

each R₁ and R₂ are independently C₁₋₁₀ alkyl, aryl, benzyl or heteroarylC₀₋₅ alkyl each R₁ and R₂ are optionally substituted with one or more amine, C₁₋₅alkyl, C₁₋₅alkoxy, oxo or halogen;

or R₁ and R₂ form a 5-6 membered heterocyclic or heteroaryl ring each optionally substituted with one or more amine, C₁₋₅alkyl, C₁₋₅alkoxy, oxo or halogen;

Xc is chosen from bond, —O—, —N(R_{b})—, —S(O)ₘ—;

R₃ is C₁₋₁₀ alkyl chain branched or unbranched optionally substituted with one or more R_{b},
or R₃ is the group:
   —(CH₂)ₛ-L-R₆, wherein L is chosen from a bond, —NH—C(O)—, —O—C(O)—, —C(O)— and —S(O)ₘ—, and wherein said group is optionally substituted by one or more R_{b};
wherein R₆ is independently chosen from hydrogen, hydroxy, alkyl, alkoxy, alkylthio, arylC₀₋₅ alkyl, aryloxyC₀₋₅ alkyl, heteroarylC₀₋₅ alkyl, cycloalkylC₀₋₅ alkyl, heterocyclylC₀₋₅ alkyl and amino said amino is optionally mono- or di-substituted by acyl, alkyl, alkoxycarbonyl, cycloalkylC₀₋₅ alkyl, arylC₀₋₅ alkyl, heteroarylC₀₋₅ alkyl or heterocyclylC₀₋₅ alkyl;

R₄, covalently attached at the indicated 4-, 5- or 6-position of the formula (I), is chosen from hydrogen, alkyl, alkoxy and halogen;

R₅ is chosen from carbocycle, heterocycle and heteroaryl each optionally substituted with one or more Rₐ;

each Rₐ or R_{b} are independently chosen from hydrogen, alkyl, alkenyl, alkynyl, cycloalkyl, cycloalkenyl, aryl, arylalkyl, aryloxy, alkoxy, alkylthio, acyl, alkoxycarbonyl, acyloxy, acylamino, sulphonylamino, aminosulfonyl, alkylsulfonyl, carboxy, carboxamide, oxo, hydroxy, halogen, trifluoromethyl, nitro, nitrile and amino optionally mono-or-di-substituted by alkyl, acyl or alkoxycarbonyl, wherein any of the above Rₐ or R_{b} are optionally halogenated where possible;

n is 1 or 2;

s is 1-10;

m is 0, 1 or 2;

or the pharmaceutically acceptable salts, esters, acids, isomers or tautomers thereof.

In yet another embodiment, there is provided a compound of the formula (I) as described immediately above and wherein:

R₄ is chosen from hydrogen and C₁₋₃ alkyl;

R₅ is a group chosen from:

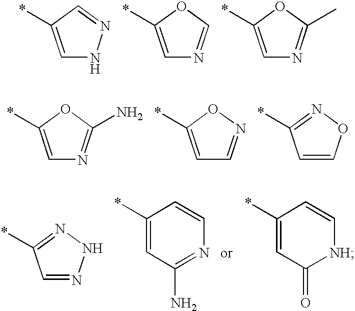

R₁ and R₂ are independently chosen from hydrogen, methyl, ethyl, propyl, isopropyl, butyl, tert-butyl,

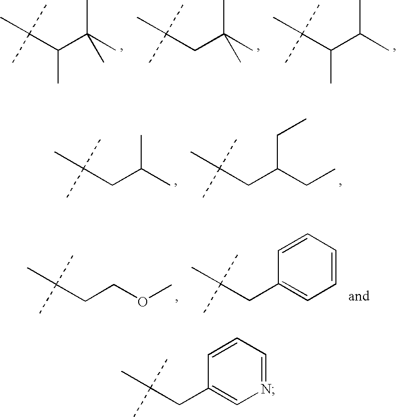

or R₁ and R₂ form following rings

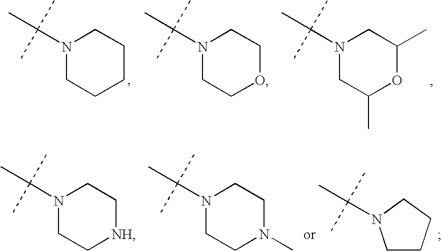

R₅ is chosen from C₄₋₈ cycloalkenyl, C₄₋₈ cycloalkyl, phenyl, naphthyl, benzothiophenyl, benzodioxolyl, quinolinyl, indolyl, thiazolyl, thienyl, furanyl, isoxazolyl, oxazolyl, imidazolyl, thiadiazolyl, pyrazolyl, pyrazinyl and pyridinyl each is optionally substituted with one or more Rₐ;

R₆ is independently chosen from hydroxy, C₁₋₅ alkyl, C₁₋₅ alkoxy, phenyl, benzyl, phenethyl, heteroarylC₀₋₅ alkyl, heterocyclylC₀₋₅ alkyl, C₃₋₇ cycloalkyl and amino said amino is optionally mono- or di-substituted by C₁₋₅ acyl, C₁₋₅ alkyl, C₁₋₅ alkoxycarbonyl, arylC₀₋₅ alkyl or heteroarylC₀₋₅ alkyl;
and wherein each recited heteroaryl in this paragraph is chosen from thienyl, furanyl, isoxazolyl, oxazolyl, thiazolyl, thiadiazolyl, tetrazolyl, pyrazolyl, pyrrolyl and imidazolyl, each optionally substituted by R_{b};

s is 1-6.

In another embodiment, there is provided a compound in accordance with the first generic embodiment above and wherein:

Q is

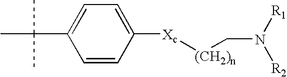

R₅ is

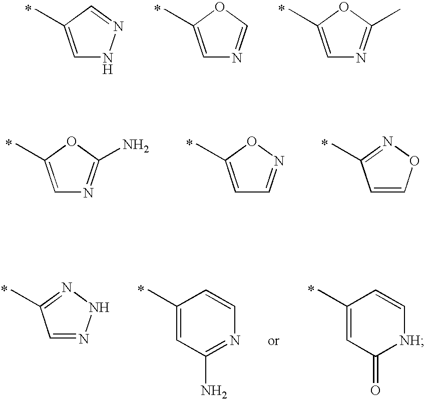

R₄ is methyl or hydrogen;

R₃ is

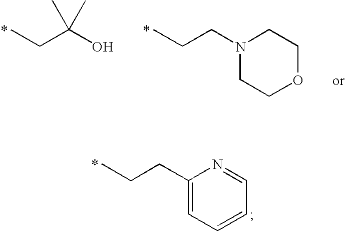

R₁ and R₂ are independently chosen from hydrogen, methyl, ethyl, propyl, isopropyl, butyl, tert-butyl,

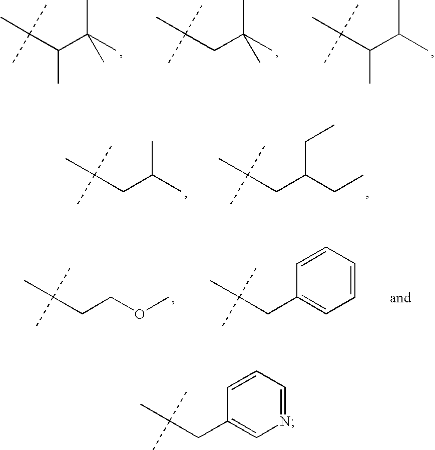

or R₁ and R₂ form following rings

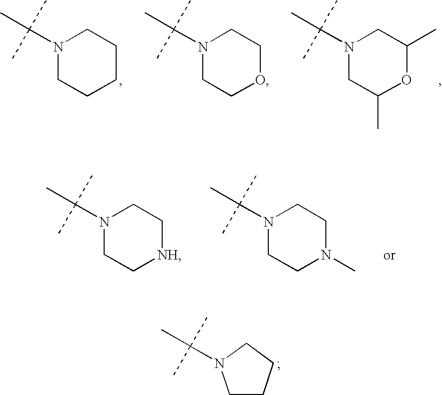

Xc is chosen from bond, —O—, —N(R_{b})—, —S(O)ₘ—.

In another embodiment there is provided representative compounds of the invention which can be made in accordance with the general schemes and working examples presented below:

TABLE I

| Structure | Name |
|---|---|
| 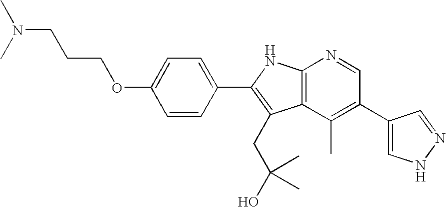 | 1-[2-[4-(3-Dimethylamino-propoxy)-phenyl]-4-methyl-5-(1H-pyrazol-4-yl)-1H-pyrrolo[2,3-b]pyridin-3-yl]-2-methyl-propan-2-ol |
| 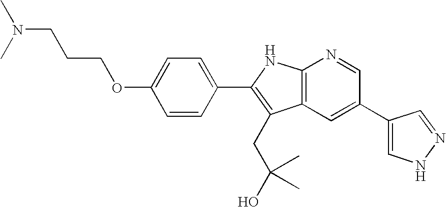 | 1-[2-[4-(3-Dimethylamino-propoxy)-phenyl]-5-(1H-pyrazol-4-yl)-1H-pyrrolo[2,3-b]pyridin-3-yl]-2-methyl-propan-2-ol |
| 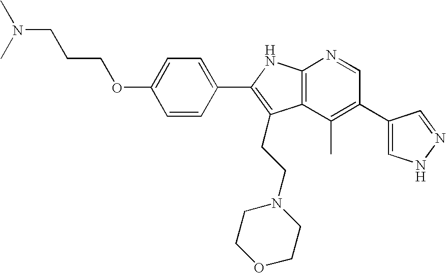 | Dimethyl-(3-{4-[4-methyl-3-(2-morpholin-4-yl-ethyl)-5-(1H-pyrazol-4-yl)-1H-pyrrolo[2,3-b]pyridin-2-yl]-phenoxy}-propyl)-amine |
| 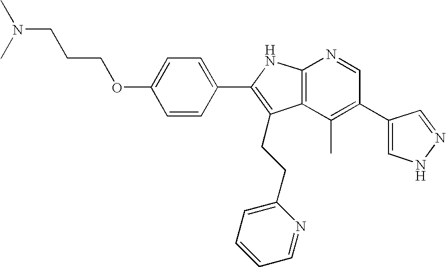 | Dimethyl-(3-{4-[4-methyl-5-(1H-pyrazol-4-yl)-3-(2-pyridin-2-yl-ethyl)-1H-pyrrolo[2,3-b]pyridin-2-yl]-phenoxy}-propyl)-amine |
| 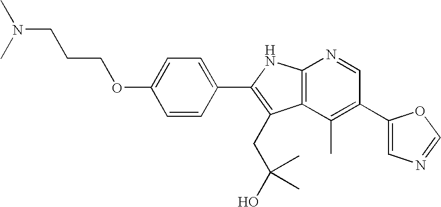 | 1-{2-[4-(3-Dimethylamino-propoxy)-phenyl]-4-methyl-5-oxazol-5-yl-1H-pyrrolo[2,3-b]pyridin-3-yl}-2-methyl-propan-2-ol |
| 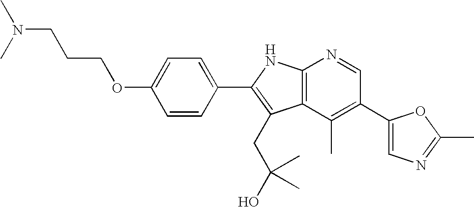 | 1-[2-[4-(3-Dimethylamino-propoxy)-phenyl]-4-methyl-5-(2-methyl-oxazol-5-yl)-1H-pyrrolo[2,3-b]pyridin-3-yl]-2-methyl-propan-2-ol |
| 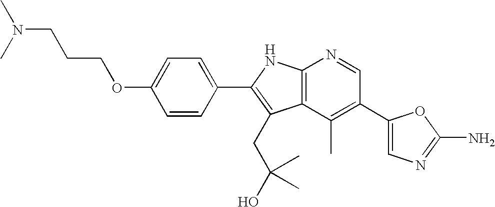 | 1-{5-(2-Amino-oxazol-5-yl)-2-[4-(3-dimethylamino-propoxy)-phenyl]-4-methyl-1H-pyrrolo[2,3-b]pyridin-3-yl}-2-methyl-propan-2-ol |
| 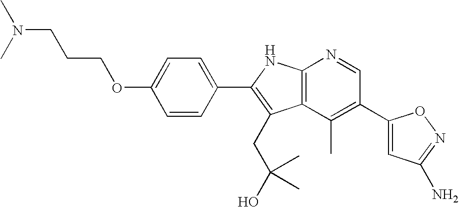 | 1-{5-(3-Amino-isoxazol-5-yl)-2-[4-(3-dimethylamino-propoxy)-phenyl]-4-methyl-1H-pyrrolo[2,3-b]pyridin-3-yl}-2-methyl-propan-2-ol |
| 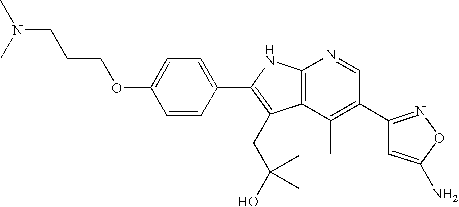 | 1-{5-(5-Amino-isoxazol-3-yl)-2-[4-(3-dimethylamino-propoxy)-phenyl]-4-methyl-1H-pyrrolo[2,3-b]pyridin-3-yl}-2-methyl-propan-2-ol |
| 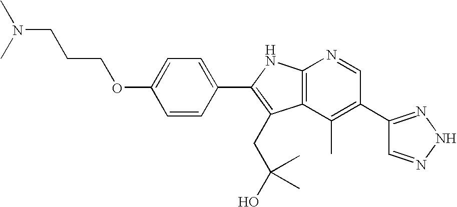 | 1-[2-[4-(3-Dimethylamino-propoxy)-phenyl]-4-methyl-5-(2H-[1,2,3]triazol-4-yl)-1H-pyrrolo[2,3-b]pyridin-3-yl]-2-methyl-propan-2-ol |
| 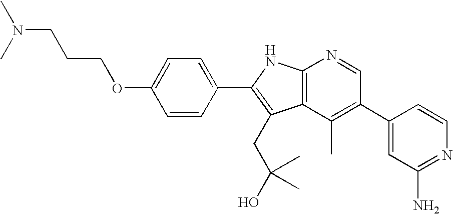 | 1-{5-(2-Amino-pyridin-4-yl)-2-[4-(3-dimethylamino-propoxy)-phenyl]-4-methyl-1H-pyrrolo[2,3-b]pyridin-3-yl}-2-methyl-propan-2-ol |
| 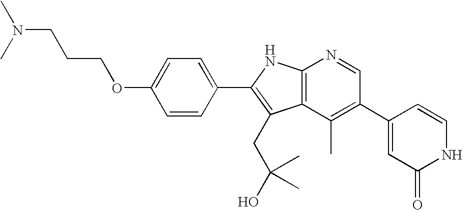 | 4-[2-[4-(3-Dimethylamino-propoxy)-phenyl]-3-(2-hydroxy-2-methyl-propyl)-4-methyl-1H-pyrrolo[2,3-b]pyridin-5-yl]-1H-pyridin-2-one |
| 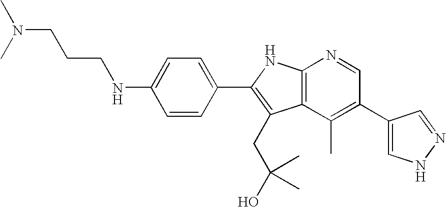 | 1-[2-[4-(3-Dimethylamino-propylamino)-phenyl]-4-methyl-5-(1H-pyrazol-4-yl)-1H-pyrrolo[2,3-b]pyridin-3-yl]-2-methyl-propan-2-ol |
| 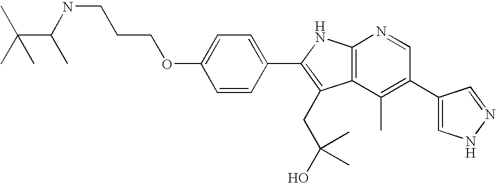 | 2-Methyl-1-(4-methyl-5-(1H-pyrazol-4-yl)-2-{4-[3-(1,2,2-trimethyl-propylamino)-propoxy]-phenyl}-1H-pyrrolo[2,3-b]pyridin-3-yl)-propan-2-ol |
| 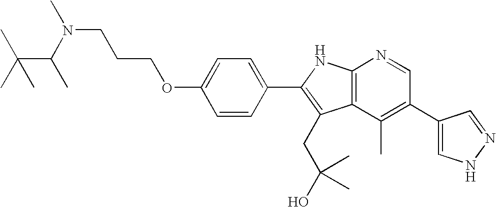 | 2-Methyl-1-[4-methyl-2-(4-{3-[methyl-(1,2,2-trimethyl-propyl)-amino]-propoxy}-phenyl)-5-(1H-pyrazol-4-yl)-1H-pyrrolo[2,3-b]pyridin-3-yl]-propan-2-ol |
| 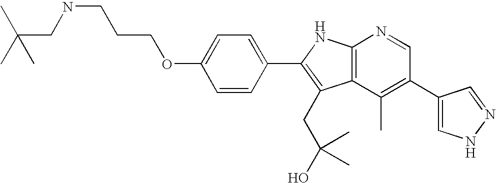 | 1-[2-{4-[3-(2,2-Dimethyl-propylamino)-propoxy]-phenyl}-4-methyl-5-(1H-pyrazol-4-yl)-1H-pyrrolo[2,3-b]pyridin-3-yl]-2-methyl-propan-2-ol |
| 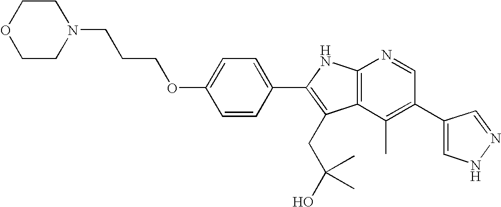 | 2-Methyl-1-[4-methyl-2-[4-(3-morpholin-4-yl-propoxy)-phenyl]-5-(1H-pyrazol-4-yl)-1H-pyrrolo[2,3-b]pyridin-3-yl]-propan-2-ol |
| 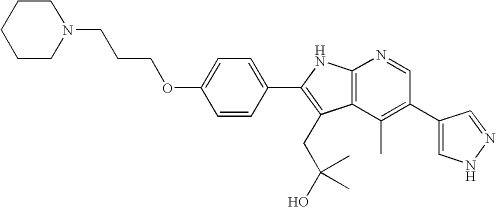 | 2-Methyl-1-[4-methyl-2-[4-(3-piperidin-1-yl-propoxy)-phenyl]-5-(1H-pyrazol-4-yl)-1H-pyrrolo[2,3-b]pyridin-3-yl]-propan-2-ol |
| 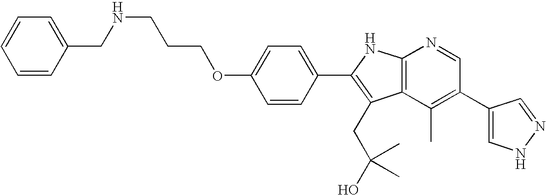 | 1-[2-[4-(3-Benzylamino-propoxy)-phenyl]-4-methyl-5-(1H-pyrazol-4-yl)-1H-pyrrolo[2,3-b]pyridin-3-yl]-2-methyl-propan-2-ol |
| 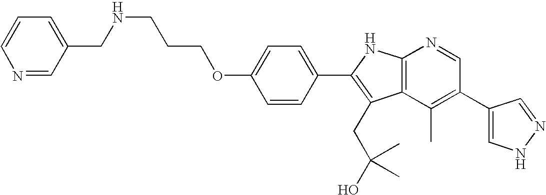 | 2-Methyl-1-[4-methyl-5-(1H-pyrazol-4-yl)-2-(4-{3-[(pyridin-3-ylmethyl)-amino]-propoxy}-phenyl)-1H-pyrrolo[2,3-b]pyridin-3-yl]-propan-2-ol |
| 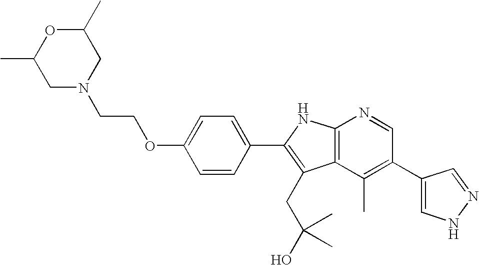 | 1-[2-{4-[2-(2,6-Dimethyl-morpholin-4-yl)-ethoxy]-phenyl}-4-methyl-5-(1H-pyrazol-4-yl)-1H-pyrrolo[2,3-b]pyridin-3-yl]-2-methyl-propan-2-ol |
| 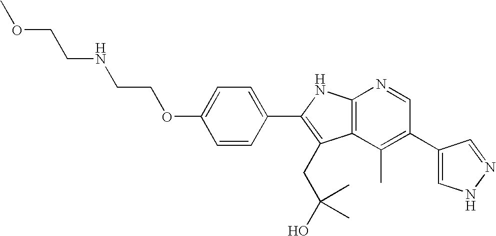 | 1-[2-{4-[2-(2-Methoxy-ethylamino)-ethoxy]-phenyl}-4-methyl-5-(1H-pyrazol-4-yl)-1H-pyrrolo[2,3-b]pyridin-3-yl]-2-methyl-propan-2-ol |

or the pharmaceutically acceptable salts thereof.

Any of the aforementioned embodiments disclosed above may have Rₐ or R_{b} also being defined as azido. Such compounds are useful as photolabeling probes and include, for example, 4-azido-phenyl moieties.

In all the compounds disclosed herein above in this application, in the event the nomenclature is in conflict with the structure, it shall be understood that the compound is defined by the structure.

The invention includes the use of any compounds described above containing one or more asymmetric carbon atoms which may occur as racemates and racemic mixtures, single enantiomers, diastereomeric mixtures and individual diastereomers. All such isomeric forms of these compounds are expressly included in the present invention. Each stereogenic carbon may be in the R or S configuration, or a combination of configurations.

Of particular importance according to the invention are compounds of formula (I), wherein Q, R₁, R₂, R₃, R₄, and R₅ have the meaning indicated, for use as pharmaceutical compositions with an anti-Tec kinase activity.

The invention also relates to the use of a compound of formula (I), wherein Q, R₁, R₂, R₃, R₄ and R₅ have the meaning indicated, for preparing a pharmaceutical composition for the treatment and/or prevention of a Tec kinase mediated disease or condition.

The invention also relates to pharmaceutical preparations, containing as active substance one or more compounds of formula (I), wherein Q, R₁, R₂, R₃, R₄ and R₅ have the meanings indicated, or the pharmaceutically acceptable derivatives thereof, optionally combined with conventional excipients and/or carriers.

Compounds of the invention also include their isotopically-labelled forms. An isotopically-labelled form of an active agent of a combination of the present invention is identical to said active agent but for the fact that one or more atoms of said active agent have been replaced by an atom or atoms having an atomic mass or mass number different from the atomic mass or mass number of said atom which is usually found in nature. Examples of isotopes which are readily available commercially and which can be incorporated into an active agent of a combination of the present invention in accordance with well established procedures, include isotopes of hydrogen, carbon, nitrogen, oxygen, phosphorous, fluorine and chlorine, e.g. ²H, ³H, ¹³C, ¹⁴C, ¹⁵N, ¹⁸O, ¹⁷O, ³¹P, ³²P, ³⁵S, ¹⁸F, and ³⁶Cl, respectively. An active agent of a combination of the present invention, a prodrug thereof, or a pharmaceutically acceptable salt of either which contains one or more of the above-mentioned isotopes and/or other isotopes of other atoms is contemplated to be within the scope of the present invention.

Some of the compounds of formula (I) can exist in more than one tautomeric form. The invention includes methods using all such tautomers.

All terms as used herein in this specification, unless otherwise stated, shall be understood in their ordinary meaning as known in the art.

Alkyl, alkenyl, alkynyl, alkoxy, alkylthio, acyl, alkoxycarbonyl, acyloxy, acylamino, alkylsulfonyl and all other alkyl containing groups shall be understood unless otherwise specified as being C1-10, branched or unbranched where structurally possible, and optionally partially or fully halogenated. For ‘C₀₋ₙ alkyl’, where n is an integer 1,2,3 etc, shall be understood to be a bond when the definition is ‘C₀’, and alkyl when n is greater than or equal to 1. Other more specific definitions are as follows:

BOC or t-BOC is tertiary-butoxycarbonyl.
t-Bu is tertiary-butyl.
DMF is dimethylformamide.
EtOAc is ethyl acetate.
EtOH and MeOH are ethanol and methanol, respectively.
TFA is trifluoroacetic acid.
THF is tetrahydrofuran.
DMSO is dimethylsulfoxide.
TBTU is O-(1H-benzotriazol-1-yl)-N,N,N′,N′-tetramethyluronium tetrafluoroborate.
FMOC is 9-fluorenylmethoxycarbonyl.

The term “aroyl” as used in the present specification shall be understood to mean “benzoyl” or “naphthoyl”.

The term “carbocycle” shall be understood to mean an aliphatic hydrocarbon radical containing from three to twelve carbon atoms. Carbocycles include hydrocarbon rings containing from three to ten carbon atoms. These carbocycles may be either aromatic and non-aromatic ring systems, and optionally or fully halogenated. The non-aromatic ring systems may be mono- or polyunsaturated. Preferred carbocycles include but are not limited to cyclopropyl, cyclobutyl, cyclopentyl, cyclopentenyl, cyclohexyl, cyclohexenyl, cycloheptanyl, cycloheptenyl, phenyl, indanyl, indenyl, benzocyclobutanyl, dihydronaphthyl, tetrahydronaphthyl, naphthyl, decahydronaphthyl, benzocycloheptanyl and benzocycloheptenyl. Certain terms for cycloalkyl such as cyclobutanyl and cyclobutyl shall be used interchangeably.

The term “heterocycle” refers to a stable nonaromatic 4-8 membered (but preferably, 5 or 6 membered) monocyclic or nonaromatic 8-11 membered bicyclic heterocycle radical which may be either saturated or unsaturated. Each heterocycle consists of carbon atoms and one or more, preferably from 1 to 4 heteroatoms selected from nitrogen, oxygen and sulfur. The heterocycle may be attached by any atom of the cycle, which results in the creation of a stable structure. Unless otherwise stated, heterocycles include but are not limited to, pyrrolidinyl, morpholinyl, thiomorpholinyl, dioxalanyl, piperidinyl, piperazinyl, aziridinyl and tetrahydrofuranyl.

The term “heteroaryl” shall be understood to mean an aromatic 5-8 membered monocyclic or 8-11 membered bicyclic ring containing 1-4 heteroatoms such as N,O and S. Unless otherwise stated, such heteroaryls include but are not limited to thienyl, furanyl, isoxazolyl, oxazolyl, thiazolyl, thiadiazolyl, tetrazolyl, pyrazolyl, pyrrolyl, imidazolyl, pyridinyl, pyrimidinyl, pyrazinyl, pyridazinyl, pyranyl, quinoxalinyl, indolyl, benzimidazolyl, benzoxazolyl, benzothiazolyl, benzothienyl, quinolinyl, quinazolinyl and indazolyl.

The term “heteroatom” as used herein shall be understood to mean atoms other than carbon such as O, N, S and P.

In all alkyl groups or carbon chains within cycloalkyl groups, where one or more carbon atoms are optionally replaced by heteroatoms: O, S or N, it shall be understood that if N is not substituted then it is NH, it shall also be understood that the heteroatoms may replace either terminal carbon atoms or internal carbon atoms within a branched or unbranched carbon chain.

Substitution on a carbon such as a methylene carbon by groups such as oxo result in definitions such as: alkoxycarbonyl, acyl, and amido, or if substituted on a ring can, for example, replace a methylene group —CH₂— with a carbonyl>C═O.

The term “aryl” as used herein shall be understood to mean aromatic carbocycle or heteroaryl as defined herein. Each aryl or heteroaryl unless otherwise specified includes its partially or fully hydrogenated derivative. For example, quinolinyl may include decahydroquinolinyl and tetrahydroquinolinyl, naphthyl may include its hydrogenated derivatives such as tetrahydranaphthyl. Each may be partially or fully halogenated. Other partially or fully hydrogenated derivatives of the aryl and heteroaryl compounds described herein will be apparent to one of ordinary skill in the art.

Terms which are analogs of the above cyclic moieties such as aryloxy or heteroaryl amine shall be understood to mean an aryl, heteroaryl, heterocycle as defined above attached to it's respective functional group.

As used herein, “nitrogen” and “sulfur” include any oxidized form of nitrogen and sulfur and the quaternized form of any basic nitrogen. For example, for an alkylthio radical such as —S—C₁₋₆ alkyl, unless otherwise specified, this shall be understood to include —S(O)—C₁₋₆ alkyl and —S(O)₂—C₁₋₆ alkyl.

The term “halogen” as used in the present specification shall be understood to mean bromine, chlorine, fluorine or iodine. The definitions “partially or fully halogenated” “substituted by one or more halogen atoms” includes for example, mono, di or tri halo derivatives on one or more carbon atoms. A non-limiting example would be a halogenated alkyl such as —CH₂CHF₂, —CF₃ etc.

The compounds of the invention are only those which are contemplated to be ‘chemically stable’ as will be appreciated by those skilled in the art. For example, a compound which would have a ‘dangling valency’, or a ‘carbanion’ are not compounds contemplated by the inventive methods disclosed herein.

The term “patient” refers to a warm-blooded mammal and preferably, a human.

The invention includes pharmaceutically acceptable derivatives of compounds of formula (I). A “pharmaceutically acceptable derivative” refers to any pharmaceutically acceptable salt or ester, or any other compound which, upon administration to a patient, is capable of providing (directly or indirectly) a compound useful for the invention, or a pharmacologically active metabolite or pharmacologically active residue thereof. A pharmacologically active metabolite shall be understood to mean any compound of the invention capable of being metabolized enzymatically or chemically. This includes, for example, hydroxylated or oxidized derivative compounds of the formula (I).

Pharmaceutically acceptable salts include those derived from pharmaceutically acceptable inorganic and organic acids and bases. Examples of suitable acids include hydrochloric, hydrobromic, sulfuric, nitric, perchloric, fumaric, maleic, phosphoric, glycolic, lactic, salicylic, succinic, toluene-p-sulfuric, tartaric, acetic, citric, methanesulfonic, formic, benzoic, malonic, naphthalene-2-sulfuric and benzenesulfonic acids. Other acids, such as oxalic acid, while not themselves pharmaceutically acceptable, may be employed in the preparation of salts useful as intermediates in obtaining the compounds and their pharmaceutically acceptable acid addition salts. Salts derived from appropriate bases include alkali metal (e.g., sodium), alkaline earth metal (e.g., magnesium), ammonium and N—(C₁-C₄ alkyl)₄⁺ salts.

In addition, within the scope of the invention is use of prodrugs of compounds of the formula (I). Prodrugs include those compounds that, upon simple chemical transformation, are modified to produce compounds of the invention. Simple chemical transformations include hydrolysis, oxidation and reduction. Specifically, when a prodrug is administered to a patient, the prodrug may be transformed into a compound disclosed herein above, thereby imparting the desired pharmacological effect.

### Methods of Therapeutic Use

The compounds of the invention are effective inhibitors of Tec kinase family activity, especially of Itk. Therefore, in one embodiment of the invention, there is provided methods of treating immunological disorders using compounds of the invention. In another embodiment, there is provided methods of treating inflammatory disorders using compounds of the invention. In yet another embodiment, there is provided methods of treating allergic disorders using compounds of the invention. In yet still another embodiment, there is provided methods of enhancing memory cell generation for vaccines using compounds of the invention. In a further embodiment, there is provided methods of treating cell proliferative disorders using compounds of the invention.

Without wishing to be bound by theory, the compounds of this invention modulate T cell and mast cell activation via effective inhibition of Itk. The inhibition of T cell activation is therapeutically useful for selectively suppressing immune function. Thus, the inhibition of Itk is an attractive means for preventing and treating a variety of immune disorders, including inflammatory diseases, autoimmune diseases, organ and bone marrow transplant rejection and other disorders associated with T cell mediated immune response. In particular, the compounds of the invention may be used to prevent or treat acute or chronic inflammation, allergies, contact dermatitis, psoriasis, rheumatoid arthritis, multiple sclerosis, type 1 diabetes, inflammatory bowel disease, Guillain-Barre syndrome, Crohn's disease, ulcerative colitis, cancer, graft versus host disease (and other forms of organ or bone marrow transplant rejection) and lupus erythematosus.

The compounds of the invention are also effective inhibitors of Tec family kinases other than Itk including Txk, Tec, Btk, and Bmx and would thus be useful in treating diseases associated with the activity of one or more of these Tec family kinases.

Inhibitors of mast cell activation and degranulation block the release of allergic and pro-inflammatory mediators and cytokines. Thus inhibitors of Itk have potential utility in treating inflammatory and allergic disorders, including asthma, chronic obstructive pulmonary disease (COPD), adult respiratory distress syndrome (ARDS), bronchitis, conjunctivitis, dermatitis and allergic rhinitis. Other disorders associated with T cell or mast cell mediated immune response will be evident to those of ordinary skill in the art and can also be treated with the compounds and compositions of this invention.

Inhibitors of Itk and other Tec family kinases have potential utility in combination with other therapies for the treatment of immune, inflammatory, proliferative, and allergic disorders. Examples, though not all encompassing, include co-administration with steroids, leukotriene antagonists, anti-histamines, cyclosporin, or rapamycin.

One strategy to improve vaccination methods is to increase the number of memory T cells generated. As described in the Background, in the absence of Itk in mice, increased numbers of memory cells are generated. Thus, within the scope of the invention is the use of the present compounds in the formulation of improved vaccines that generate increased numbers of memory T cells.

For therapeutic use, the compounds of the invention may be administered in any conventional dosage form in any conventional manner. Routes of administration include, but are not limited to, intravenously, intramuscularly, subcutaneously, intrasynovially, by infusion, sublingually, transdermally, orally, topically or by inhalation. The preferred modes of administration are oral and intravenous.

The compounds of this invention may be administered alone or in combination with adjuvants that enhance stability of the inhibitors, facilitate administration of pharmaceutic compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies. Compounds of the invention may be physically combined with the conventional therapeutics or other adjuvants into a single pharmaceutical composition. Advantageously, the compounds may then be administered together in a single dosage form. In some embodiments, the pharmaceutical compositions comprising such combinations of compounds contain at least about 5%, but more preferably at least about 20%, of a compound of formula (I) (w/w) or a combination thereof. The optimum percentage (w/w) of a compound of the invention may vary and is within the purview of those skilled in the art. Alternatively, the compounds may be administered separately (either serially or in parallel). Separate dosing allows for greater flexibility in the dosing regime.

As mentioned above, dosage forms of the compounds of this invention include pharmaceutically acceptable carriers and adjuvants known to those of ordinary skill in the art. These carriers and adjuvants include, for example, ion exchangers, alumina, aluminum stearate, lecithin, serum proteins, buffer substances, water, salts or electrolytes and cellulose-based substances. Preferred dosage forms include, tablet, capsule, caplet, liquid, solution, suspension, emulsion, lozenges, syrup, reconstitutable powder, granule, suppository and transdermal patch. Methods for preparing such dosage forms are known (see, for example, H. C. Ansel and N. G. Popovish, Pharmaceutical Dosage Forms and Drug Delivery Systems, 5th ed., Lea and Febiger (1990)). Dosage levels and requirements are well-recognized in the art and may be selected by those of ordinary skill in the art from available methods and techniques suitable for a particular patient. In some embodiments, dosage levels range from about 1-1000 mg/dose for a 70 kg patient. Although one dose per day may be sufficient, up to 5 doses per day may be given. For oral doses, up to 2000 mg/day may be required. As the skilled artisan will appreciate, lower or higher doses may be required depending on particular factors. For instance, specific dosage and treatment regimens will depend on factors such as the patient's general health profile, the severity and course of the patient's disorder or disposition thereto, and the judgment of the treating physician.

### BIOLOGICAL ACTIVITY

### Tec Family Kinase Assay

Itk, Txk, Tec, Btk, and Bmx are purified as a GST-fusion protein. The kinase activity is measured using DELFIA (Dissociation Enhanced Lanthanide Fluoroimmunoassay) which utilizes europium chelate-labeled anti-phosphotyrosine antibodies to detect phosphate transfer to a random polymer, poly Glu₄: Tyr₁ (PGTYR). The screen is run on the Zymark Allegro robot system to dispense reagents, buffers and samples for assay, and also to wash and read plates. The kinase assay is performed in kinase assay buffer (50 mM HEPES, pH 7.0, 25 mM MgCl₂, 5 mM MnCl₂, 50 mM KCl, 100 μM Na₃VO₄, 0.2% BSA, 0.01% CHAPS, 200 μM TCEP). Test samples initially dissolved in DMSO at 1 mg/mL, are pre-diluted for dose response (10 doses with starting final concentration of 3 μg/mL, 1 to 3 serial dilutions) with the assay buffer in 96-well polypropylene microtiter plates. A 50 μL volume/well of a mixture of substrates containing ATP (final ATP concentration in each kinase assay is equal to its apparent ATP Kₘ) and 3.6 ng/μL PGTYR-biotin (CIS Bio International) in kinase buffer is added to neutravidin coated 96-well white plate (PIERCE), followed by 25 μL/well test sample solution and 25 μL/well of diluted enzyme (1-7 nM final conc.). Background wells are incubated with buffer, rather than 25 μL enzyme. The assay plates are incubated for 30 min at room temperature. Following incubation, the assay plates are washed three times with 250 μL DELFIA wash buffer. A 100 μL aliquot of 1 nM europium-labeled anti-phosphotyrosine (Eu³⁺-PT66, Wallac CR04-100) diluted in DELFIA assay buffer is added to each well and incubated for 30 min at room temperature. Upon completion of the incubation, the plate is washed four times with 250 μL of wash buffer and 100 μL of DELFIA Enhancement Solution (Wallac) is added to each well. After 15 min of longer, time-resolved fluorescence is measured (excitation at 360 nm, emission at 620 nm) after a delay time of 250 μs.

Preferred compounds of the invention have an activity of 1 microMolar or less.

In order that this invention be more fully understood, the following examples are set forth. These examples are for the purpose of illustrating preferred embodiments of this invention, and are not to be construed as limiting the scope of the invention in any way.

The examples which follow are illustrative and, as recognized by one skilled in the art, particular reagents or conditions could be modified as needed for individual compounds without undue experimentation. Starting materials used in the schemes below are either commercially available or easily prepared from commercially available materials by those skilled in the art.

### General Synthetic Methods

The invention also provides processes for making compounds of formula I. Intermediates used in the preparation of compounds of the invention are either commercially available or readily prepared by methods known to those skilled in the art. Further reference in this regard may be made to WO 03/041708 corresponding to US publication US 2003-0144281, and PCT application PCT/U503/24024 corresponding to US publication 2005-0203158.

Compounds of formula (I) may be prepared as illustrated in Scheme 1. An optionally substituted aminobromopyridine (II) may be arylated by reaction with R₅B(OH)₂ in the presence of a palladium catalyst using cross coupling chemistry well known in the art (see for example, A. Suzuki, J. Organomet. Chem. 1999, 576, 147) to provide intermediate (III), where R³, R⁴, R⁵R or Q shall have the meaning given for the formula I of the invention described herein above.

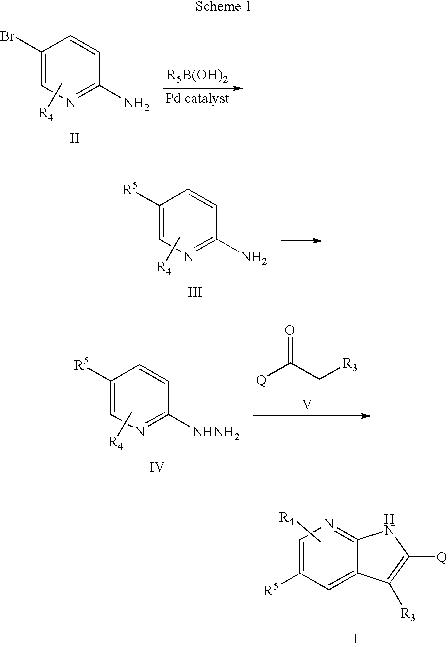

Intermediate III may be converted to the 2-hydrazinopyridine intermediate IV using conditions known in the art (see for example A. Boido et al., Farmaco, 1989, 44, 279 or K. Mogilaiah et al., Indian J. Chem. Sect. B: Org. Incl. Med. Chem., 2002, 41, 1894).

Reaction of a compound of formula (IV) with a compound of formula (V) provides a compound of formula (I). The process to convert the intermediate of formula (IV) to (I) may be carried out by heating (IV) and (V) together at a suitable temperature and preferably in an inert atmosphere, optionally in the presence of inert solvent. Preferably the reaction is carried out at temperature between 100° C. and 250° C., preferably in the absence of a solvent. Suitable reaction times are generally from 5 minutes to 3 hours. A Similar reaction protocol is reported by P. Aadal Nielsen et al., (WO2004/016609). The resulting compound may be further modified by methods known in the art to give additional compounds of formula (I).

### Synthetic Example

### Example 1

### Synthesis of 4-(4,4,5,5-Tetramethyl-[1,3,2]dioxaborolan-2-yl)-1-trityl-1H-pyrazole

This example illustrates the synthesis of an intermediate which may be used in the preparation of compounds of formula (I).

A solution of 150 g (2.2 mol) of pyrazole and 703 g (4.4 mol, 2.0 eq.) of bromine in 3 L of water was refluxed for 1 h. The resulting mixture was cooled to room temperature, diluted with saturated aqueous sodium bicarbonate solution and the product was extracted into ethyl acetate. The organic layer was dried over sodium sulfate and concentrated. The residue was triturated with hexanes to give 322 g (99%) of 4-bromo-1H-pyrazole.

A solution of 150 g (1.0 mol) of 4-bromo-1H-pyrazole, 334 g (1.2 mol, 1.2 eq.) of trityl chloride, 158 g (2.0 mol, 2.0 eq.) of pyridine and 6.1 g (0.05 mol, 5 mol %) of 4-dimethylaminopyridine in 2 L of dichloromethane was stirred at room temperature for 16 h. The resulting mixture was washed with water and saturated aqueous ammonium chloride solution. The organic layer was dried over sodium sulfate and concentrated. The residue was triturated with hexanes to give 380 g (96%) of 4-bromo-1-trityl-1H-pyrazole.

A stirred mixture of 5.0 g (12.8 mmol) of 4-bromo-1-trityl-1H-pyrazole, 9.8 g (38.6 mmol, 3.0 eq.) of bis(pinacolato)diboron, 1.0 g (1.3 mmol, 10 mol %) of PdCl₂(dppf) and 6.3 g (64.2 mmol, 5.0 eq) of potassium acetate in DMF (30 mL) was heated at 80° C. for 16 h. The resulting mixture was diluted with water, and the product was extracted with dichloromethane. The organic layer was washed with brine, dried over sodium sulfate, and purified by flash chromatography to give 5.1 g (91%) of 4-(4,4,5,5-tetramethyl-[1,3,2]dioxaborolan-2-yl)-1-trityl-1H-pyrazole.

### Example 2

### Synthesis of 1-[2-[4-(3-Dimethylamino-propoxy)-phenyl]-4-methyl-5-(1H-pyrazol-4-yl)-1H-pyrrolo[2,3-b]pyridin-3-yl]-2-methyl-propan-2-ol

The following is an illustrative example of how compounds of formula (I) may be prepared.

A mixture of 2 g (10.7 mmol) of 5-bromo-4-methyl-[2]-pyridylamine, 5.1 g (11.8 mmol, 1.1 eq) of 4-(4,4,5,5-tetramethyl-[1,3,2]dioxaborolan-2-yl)-1-trityl-1H-pyrazole and 376 mg (0.5 mmol, 5 mol %) of bis(triphenylphsophine)palladium(II) dichloride in 20 mL of DMF and 20 mL of 2M sodium carbonate solution is heated at 80° C. for 12 h. Then the reaction mixture is cooled to room temperature and the product is extracted into ethyl acetate. The organic layer is washed with brine, dried over sodium sulfate and concentrated. The residue is purified by flash chromatography to give 4-methyl-5-(1-trityl-1H-pyrazol-4-yl)-pyridin-2-ylamine.

A mixture of 1 g (2.4 mmol) of 4-methyl-5-(1-trityl-1H-pyrazol-4-yl)-pyridin-2-ylamine and 248 mg (3.6 mmol, 1.5 eq) of sodium nitrite in 25 mL of 6 M aqueous hydrochloric acid solution is stirred at 0° C. for 1 h. The mixture is treated with 1.1 g (4.8 mmol, 2.0 eq.) of tin(II) chloride dihydrate, and the resulting mixture is allowed to warm to room temperature. The mixture is stirred at this temperature for 16 h and cooled to 0° C. The reaction mixture is basified with 4 N sodium hydroxide solution. The product is extracted into ethyl acetate. The organic layer is washed with water and brine, dried over sodium sulfate and concentrated. The residue is purified by flash chromatography to give [4-methyl-5-(1H-pyrazol-4-yl)-pyridin-2-yl]-hydrazine.

A solution of 1 g (6.6 mmol) of methyl 4-hydroxybenzoate, 969 mg (7.9 mmol, 1.2 eq) of 3-dimethylamino-1-propyl chloride hydrochloride and 1.8 g (13.2 mmol, 2.0 eq) of potassium carbonate in 30 mL of DMSO is stirred at room temperature for 16 h. The mixture is diluted with water and the product is extracted into ethyl acetate. The organic layer is washed with water and brine, dried over sodium sulfate and concentrated. The residue is purified by flash chromatography to give 4-(3-dimethylamino-propoxy)-benzoic acid methyl ester.

A solution of 1 g (4.2 mmol) of 4-(3-dimethylamino-propoxy)-benzoic acid methyl ester and 352 mg (8.4 mmol, 2.0 eq) of lithium hydroxide monohydrate in 27 mL of methanol and 3 mL of water is stirred at room temperature for 16 h. The resulting mixture is acidified by acetic acid and the product is extracted into ethyl acetate. The organic layer is washed with water and brine, dried over sodium sulfate and concentrated. The residue is purified by flash chromatography to give 4-(3-dimethylaminopropoxy)-benzoic acid.

A solution of 1.0 g (4.5 mmol) of 4-(3-dimethylaminopropoxy)-benzoic acid in 20 mL of dichloromethane is treated with 2.7 mL (5.4 mmol, 1.2 eq.) of 2.0 M oxalyl chloride solution in dichloromethane and 3 drops of N,N-dimethylformamide at 0° C. The resulting mixture is allowed to warm to room temperature and stirred at this temperature for 4 h. The mixture is concentrated to give 4-(3-dimethylamino-propoxy)-benzoyl chloride, and the residue is used for the next reaction without further purification.

A solution of 1 g (11.9 mmol) of 2-methyl-but-3-yn-2-ol in 15 mL of THF is treated with 167 mg (0.24 mmol, 0.02 eq) of bis(triphenylphosphine)palladium(II) dichloride and 4.0 g (13.9 mmol, 1.2 eq) of tributyltin hydride portionwise at room temperature. After being stirred for 10 min, the reaction mixture is concentrated and the resulting residue is purified by flash chromatography to give (E)-2-methyl-4-tributylstannanyl-but-3-en-2-ol.

A solution of 1 g (4.1 mmol) of 4-(3-dimethylamino-propoxy)-benzoyl chloride, 1.9 g (5.0 mmol, 1.2 eq.) of (E)-2-methyl-4-tributylstannanyl-but-3-en-2-ol and 124 mg (0.16 mmol, 0.04 eq.) of trans-benzyl(chloro)bis(triphenylphosphin)palladium(II) in 20 mL of toluene is heated at 90° C. for 16 h in sealed tube. The resulting mixture is cooled and concentrated. The residue is purified flash chromatography to give (E)-1-[4-(3-dimethylamino-propoxy)-phenyl]-4-hydroxy-4-methyl-pent-2-en-1-one.

A mixture of 1 g (3.4 mmol) of (E)-1-[4-(3-dimethylamino-propoxy)-phenyl]-4-hydroxy-4-methyl-pent-2-en-1-one and 50 mg of 10% palladium on carbon in 50 mL of methanol is stirred at room temperature under hydrogen gas atmosphere for 16 h. The mixture is filtered through diatomaceous earth and the filtrate is concentrated to give 1-[4-(3-dimethylamino-propoxy)-phenyl]-4-hydroxy-4-methyl-pentan-1-one

A mixture of 1 g (5.3 mmol) of [4-methyl-5-(1H-pyrazol-4-yl)-pyridin-2-yl]-hydrazine, 1.6 g (5.3 mmol, 1.0 eq.) of 1-[4-(3-dimethylamino-propoxy)-phenyl]-4-hydroxy-4-methyl-pentan-1-one and 30 mg of p-toluenesulfonic acid in 50 mL of benzene is refluxed for 16 h. Water is continuously distilled off using a Dean-Stark trap. The reaction mixture is cooled and diluted with dichloromethane. The organic layer is washed with saturated aqueous sodium bicarbonate solution and brine, dried over sodium sulfate and concentrated. The residue is purified by flash chromatography to give 1-[2-[4-(3-dimethylamino-propoxy)-phenyl]-4-methyl-5-(1H-pyrazol-4-yl)-1H-pyrrolo[2,3-b]pyridin-3-yl]-2-methyl-propan-2-ol.

1. A compound of the formula (I): 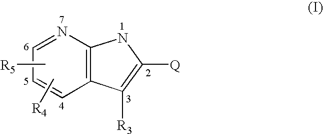
wherein:
Q is 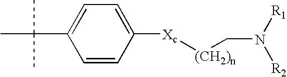
each R₁ and R₂ are independently C₁₋₁₀ alkyl, aryl, benzyl or heteroarylC₀₋₅ alkyl each R₁ and R₂ are optionally substituted with one or more amine, C₁₋₅alkyl, C₁₋₅alkoxy, oxo or halogen;
or R₁ and R₂ form a 5-6 membered heterocyclic or heteroaryl ring each optionally substituted with one or more amine, C₁₋₅alkyl, C₁₋₅alkoxy, oxo or halogen;
Xc is chosen from bond, —O—, —N(R_{b})— and —S(O)ₘ—;
R₃ is C₁₋₁₀ alkyl chain branched or unbranched optionally substituted with one or more R_{b},
or R₃ is the group:
—(CH₂)ₛ-L-R₆, wherein L is chosen from a bond, —NH—C(O)—, —O—C(O)—, —C(O)— and —S(O)ₘ—, and wherein said group is optionally substituted by one or more R_{b};
wherein R₆ is independently chosen from hydrogen, hydroxy, alkyl, alkoxy, alkylthio, arylC₀₋₅ alkyl, aryloxyC₀₋₅ alkyl, heteroarylC₀₋₅alkyl, cycloalkylC₀₋₅alkyl, heterocyclylC₀₋₅ alkyl and amino said amino is optionally mono- or di-substituted by acyl, alkyl, alkoxycarbonyl, cycloalkylC₀₋₅ alkyl, arylC₀₋₅ alkyl, heteroarylC₀₋₅ alkyl or heterocyclylC₀₋₅ alkyl;
R₄, covalently attached at the indicated 4-, 5- or 6-position of the formula (I), is chosen from hydrogen, alkyl, alkoxy and halogen;
R₅ is chosen from carbocycle, heterocycle and heteroaryl each optionally substituted with one or more Rₐ;
each Rₐ or R_{b} are independently chosen from hydrogen, alkyl, alkenyl, alkynyl, cycloalkyl, cycloalkenyl, aryl, arylalkyl, aryloxy, alkoxy, alkylthio, acyl, alkoxycarbonyl, acyloxy, acylamino, sulphonylamino, aminosulfonyl, alkylsulfonyl, carboxy, carboxamide, oxo, hydroxy, halogen, trifluoromethyl, nitro, nitrile and amino optionally mono-or-di-substituted by alkyl, acyl or alkoxycarbonyl, wherein any of the above Rₐ or R_{b} are optionally halogenated where possible;
n is 1 or 2;
s is 1-10;
m is 0, 1 or 2
or the pharmaceutically acceptable salts thereof.

2. The compound according to claim 1 and wherein
R₄ is chosen from hydrogen and C₁₋₃ alkyl;
R₅ is a group chosen from: 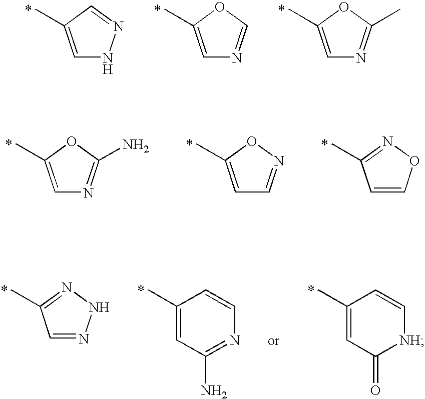
R₁ and R₂ are independently chosen from hydrogen, methyl, ethyl, propyl, isopropyl, butyl, tert-butyl, 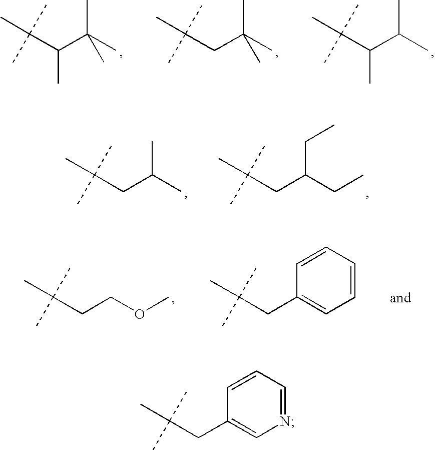
or R₁ and R₂ form following rings 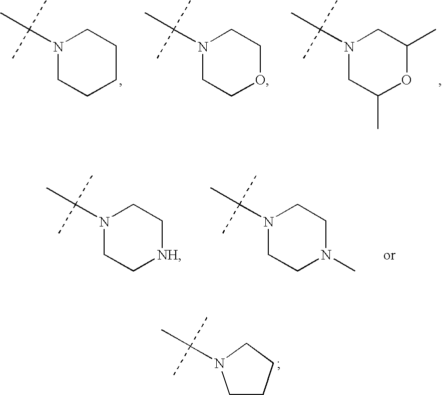
R₅ is chosen from C₄₋₈ cycloalkenyl, C₄₋₈ cycloalkyl, phenyl, naphthyl, benzothiophenyl, benzodioxolyl, quinolinyl, indolyl, thiazolyl, thienyl, furanyl, isoxazolyl, oxazolyl, imidazolyl, thiadiazolyl, pyrazolyl, pyrazinyl and pyridinyl each is optionally substituted with one or more Rₐ;
R₆ is independently chosen from hydroxy, C₁₋₅ alkyl, C₁₋₅ alkoxy, phenyl, benzyl, phenethyl, heteroarylC₀₋₅ alkyl, heterocyclylC₀₋₅ alkyl, C₃₋₇ cycloalkyl and amino said amino is optionally mono- or di-substituted by C₁₋₅ acyl, C₁₋₅ alkyl, C₁₋₅ alkoxycarbonyl, arylC₀₋₅ alkyl or heteroarylC₀₋₅ alkyl;
and wherein each recited heteroaryl in this paragraph is chosen from thienyl, furanyl, isoxazolyl, oxazolyl, thiazolyl, thiadiazolyl, tetrazolyl, pyrazolyl, pyrrolyl and imidazolyl, each optionally substituted by R_{b}; and
s is 1-6.

3. The compound according to claim 2 and wherein
Q is 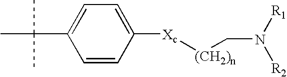
R₅ is 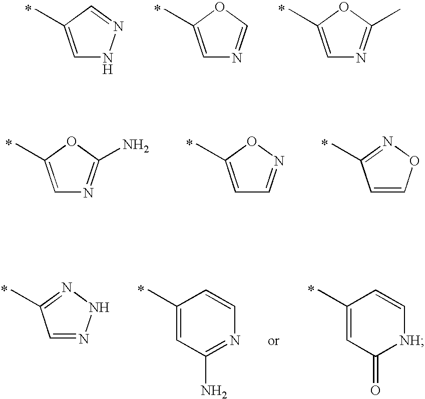
R₄ is methyl or hydrogen;
R₃ is 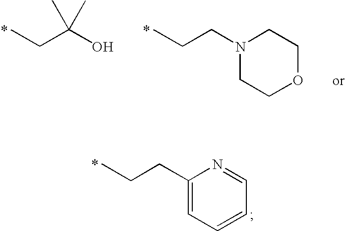
R₁ and R₂ are independently chosen from hydrogen, methyl, ethyl, propyl, isopropyl, butyl, tert-butyl, 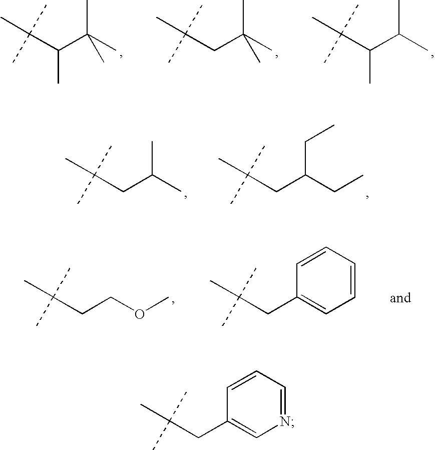
or R₁ and R₂ form following rings 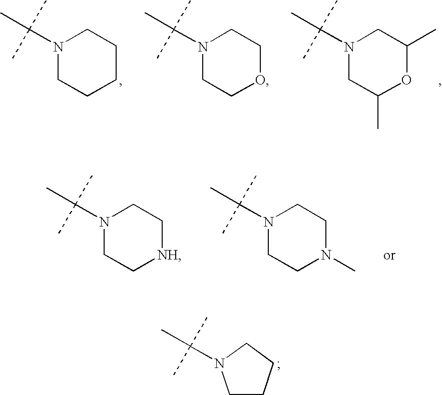
Xc is chosen from bond, —O—, —N(R_{b})— and —S(O)ₘ.

4. A compound chosen from:
1-[2-[4-(3-Dimethylamino-propoxy)-phenyl]-4-methyl-5-(1H-pyrazol-4-yl)-1H-pyrrolo[2,3-b]pyridin-3-yl]-2-methyl-propan-2-ol;
1-[2-[4-(3-Dimethylamino-propoxy)-phenyl]-5-(1H-pyrazol-4-yl)-1H-pyrrolo[2,3-b]pyridin-3-yl]-2-methyl-propan-2-ol;
Dimethyl-(3-{4-[4-methyl-3-(2-morpholin-4-yl-ethyl)-5-(1H-pyrazol-4-yl)-1H-pyrrolo[2,3-b]pyridin-2-yl]-phenoxy}-propyl)-amine;
Dimethyl-(3-{4-[4-methyl-5-(1H-pyrazol-4-yl)-3-(2-pyridin-2-yl-ethyl)-1H-pyrrolo[2,3-b]pyridin-2-yl]-phenoxy}-propyl)-amine;
1-{2-[4-(3-Dimethylamino-propoxy)-phenyl]-4-methyl-5-oxazol-5-yl-1H-pyrrolo[2,3-b]pyridin-3-yl}-2-methyl-propan-2-ol;
1-[2-[4-(3-Dimethylamino-propoxy)-phenyl]-4-methyl-5-(2-methyl-oxazol-5-yl)-1H-pyrrolo[2,3-b]pyridin-3-yl]-2-methyl-propan-2-ol;
1-{5-(2-Amino-oxazol-5-yl)-2-[4-(3-dimethylamino-propoxy)-phenyl]-4-methyl-1H-pyrrolo[2,3-b]pyridin-3-yl}-2-methyl-propan-2-ol;
1-{5-(3-Amino-isoxazol-5-yl)-2-[4-(3-dimethylamino-propoxy)-phenyl]-4-methyl-1H-pyrrolo[2,3-b]pyridin-3-yl}-2-methyl-propan-2-ol;
1-{5-(5-Amino-isoxazol-3-yl)-2-[4-(3-dimethylamino-propoxy)-phenyl]-4-methyl-1H-pyrrolo[2,3-b]pyridin-3-yl}-2-methyl-propan-2-ol;
1-[2-[4-(3-Dimethylamino-propoxy)-phenyl]-4-methyl-5-(2H-[1,2,3]triazol-4-yl)-1H-pyrrolo[2,3-b]pyridin-3-yl]-2-methyl-propan-2-ol;
1-{5-(2-Amino-pyridin-4-yl)-2-[4-(3-dimethylamino-propoxy)-phenyl]-4-methyl-1H-pyrrolo[2,3-b]pyridin-3-yl}-2-methyl-propan-2-ol;
4-[2-[4-(3-Dimethylamino-propoxy)-phenyl]-3-(2-hydroxy-2-methyl-propyl)-4-methyl-1H-pyrrolo[2,3-b]pyridin-5-yl]-1H-pyridin-2-one;
1-[2-[4-(3-Dimethylamino-propylamino)-phenyl]-4-methyl-5-(1H-pyrazol-4-yl)-1H-pyrrolo[2,3-b]pyridin-3-yl]-2-methyl-propan-2-ol;
2-Methyl-1-(4-methyl-5-(1H-pyrazol-4-yl)-2-{4-[3-(1,2,2-trimethyl-propylamino)-propoxy]-phenyl}-1H-pyrrolo[2,3-b]pyridin-3-yl)-propan-2-ol;
2-Methyl-1-[4-methyl-2-(4-{3-[methyl-(1,2,2-trimethyl-propyl)-amino]-propoxy}-phenyl)-5-(1H-pyrazol-4-yl)-1H-pyrrolo[2,3-b]pyridin-3-yl]-propan-2-ol;
1-[2-{4-[3-(2,2-Dimethyl-propylamino)-propoxy]-phenyl}-4-methyl-5-(1H-pyrazol-4-yl)-1H-pyrrolo[2,3-b]pyridin-3-yl]-2-methyl-propan-2-ol;
2-Methyl-1-[4-methyl-2-[4-(3-morpholin-4-yl-propoxy)-phenyl]-5-(1H-pyrazol-4-yl)-1H-pyrrolo[2,3-b]pyridin-3-yl]-propan-2-ol;
2-Methyl-1-[4-methyl-2-[4-(3-piperidin-1-yl-propoxy)-phenyl]-5-(1H-pyrazol-4-yl)-1H-pyrrolo[2,3-b]pyridin-3-yl]-propan-2-ol;
1-[2-[4-(3-Benzylamino-propoxy)-phenyl]-4-methyl-5-(1H-pyrazol-4-yl)-1H-pyrrolo[2,3-b]pyridin-3-yl]-2-methyl-propan-2-ol;
2-Methyl-1-[4-methyl-5-(1H-pyrazol-4-yl)-2-(4-{3-[(pyridin-3-ylmethyl)-amino]-propoxy}-phenyl)-1H-pyrrolo[2,3-b]pyridin-3-yl]-propan-2-ol;
1-[2-{4-[2-(2,6-Dimethyl-morpholin-4-yl)-ethoxy]-phenyl}-4-methyl-5-(1H-pyrazol-4-yl)-1H-pyrrolo[2,3-b]pyridin-3-yl]-2-methyl-propan-2-ol
and
1-[2-{4-[2-(2-Methoxy-ethylamino)-ethoxy]-phenyl}-4-methyl-5-(1H-pyrazol-4-yl)-1H-pyrrolo[2,3-b]pyridin-3-yl]-2-methyl-propan-2-ol
or the pharmaceutically acceptable salts thereof.

5. A method of treating a disease or condition chosen from acute or chronic inflammation, allergies, contact dermatitis, psoriasis, rheumatoid arthritis, multiple sclerosis, type 1 diabetes, inflammatory bowel disease, Guillain-Barre syndrome, Crohn's disease, ulcerative colitis, cancer, graft versus host disease and lupus erythematosus comprising administering to a patient a therapeutically effective amount of a compound according to any one of claims 1-4.

6. A method of treating a disease or condition chosen from asthma, chronic obstructive pulmonary disease (COPD), adult respiratory distress syndrome (ARDS), bronchitis, conjunctivitis, dermatitis and allergic rhinitis comprising administering to a patient a therapeutically effective amount of a compound according to any one of claims 1-4.

7. A pharmaceutical composition comprising a therapeutically effective amount of a compound according to any one of claims 1-4 and one or more pharmaceutically acceptable carriers and/or adjuvants.
